(19) United States
(12) Patent Application Publication
(10) Pub. No.: US 2011/0118750 A1
(43) Pub. Date: May 19, 2011

(54) UNIVERSAL DIRECTION MEDICAL POSITIONING STRUCTURE

(75) Inventors: Ming-Chang Wu, Guishan Shiang (TW); Jia-Jyun Hong, Guishan Shiang (TW); Hung-Sheng Tien, Guishan Shiang (TW); Chi-Bin Wu, Guishan Shiang (TW)

(73) Assignee: AccuMIS Inc., Zhonghe City (TW)

(21) Appl. No.: 12/716,292

(22) Filed: Mar. 3, 2010

(30) Foreign Application Priority Data
Nov. 18, 2009 (TW) 098139060

(51) Int. Cl. A61B 19/00

(52) U.S. Cl. 606/130

(57) ABSTRACT

Provided is a universal direction medical positioning structure, including a positioning unit, a switching head, a fixing rod, and a fixing device. The positioning unit includes a support, a connecting rod, and a spherical connecting element. The support and the spherical connecting element are connectable to two ends of the connecting rod, respectively. One end of the switching head is provided therein with a hemispherical cavity for receiving the spherical connecting element, and the other end of the switching head is coupled to the fixing rod. Upon the coupling of the fixing device and the switching head, the spherical connecting element is fixed in position to the hemispherical cavity of the switching head. Hence, the universal direction medical positioning structure is lightweight and easy to operate.

### BACKGROUND OF THE INVENTION

1. Technical Field

The present invention relates to universal direction medical positioning structures, and more particularly, to a universal direction medical positioning structure for use in a surgical navigation system.

2. Description of Related Art

As a result of injuries or spinal degeneration, the human spine (including lumbar vertebrae, thoracic vertebrae, and cervical vertebrae) is likely to encounter fracture, intervertebral disk herniation, and spondylolisthesis and thereby suffer pain when spinal nerves inside the spinal canal are compressed. The aforesaid diseases can be cured by conventional surgery that involves, for example, inserting into the human spine a front fixer, a rear fixer, a cage spacer, or an artificial intervertebral disc. In this regard, a surgical navigation system can always assist with the aforesaid surgery so as to render the aforesaid surgery smooth and successful.

Before surgery, a surgical navigation system enables a surgeon to plan the routes of surgical instruments according to the spatial information provided by the surgical navigation system. During surgery, the surgical navigation system provides the surgeon with data regarding the spatial relationship between a surgical instrument and a lesion to assist the surgeon in accurately drilling holes and inserting implants into the human body.

In general, a surgical navigation system works by integrating C-arm images into an optical positioning device and comprises of a computer, a reflection device, the optical positioning device, surgical instruments, instruments fixed in position to spinous processes, etc. Data about the spatial coordinates of the lesion are obtained by computing the position of the reflection device (attached to the instruments fixed in position to spinous processes) relative to the optical positioning device. The instruments are fixed in position to spinous processes by either clamping or insertion.

To obtain the spatial coordinates of the lesion by computing the position of the reflection device relative to the optical positioning device, the reflection device attached to the instruments fixed in position to spinous processes and the optical positioning device must be aimed at each other rather than shielded from each other so as to facilitate transmission and reception of optical signals. Thus, not only is the optical positioning device enabled to receive optical signals precisely, thereby allowing the computer to calculate the position of the lesion accurately, but also the precision in locating the surgical instruments is substantially increased. Hence, considerations should be given to improving the dexterity of motion and efficiency in timeliness of adjusting a reflection device attached to a surgical instrument fixed in position to a spinous process, particularly during surgery.

A conventional reflection device attached to instruments fixed in position to spinous processes is a three-axis (three-dimensional) structure. Hence, the conventional reflection device has three angularly adjustable switching heads whereby a surgeon can adjust the angle of the reflection device at any time. To adjust the orientation of the reflection device during surgery, the surgeon must perform the steps of: loosening the switching heads, adjusting the orientation of the reflection device, and tightening the switching heads. The aforesaid steps must be performed at least thrice on the three switching heads, which is not only time-consuming but also likely to reduce the precision of the surgical instruments and in locating the lesion as a result of difficulties presented in the adjustment of the reflection device. Also, there are few variations in conventional instruments, as conventional instruments are always fixed in position to spinous processes by either clamping or insertion and are of a standardized length.

### BRIEF SUMMARY OF THE INVENTION

The present invention provides a universal direction medical positioning structure that comprises a positioning unit with a spherical connecting element. The positioning unit can rotate freely to facilitate three-dimensional adjustment and thus eliminate the difficulty in otherwise confronting a plurality of switching heads disclosed in the prior art.

The present invention provides a universal direction medical positioning structure that uses fewer spherical connecting elements than a conventional universal direction medical positioning structure. The universal direction medical positioning structure is relatively lighter in weight, enables convenient operation, and dispenses with multiple adjustments otherwise required for the conventional universal direction medical positioning structure with a plurality of switching heads.

The present invention provides a universal direction medical positioning structure which is relatively lighter in weight and therefore unlikely to loosen when held by means of clamping or insertion. Thus, the present invention overcomes the problem that a conventional universal direction medical positioning structure has in loosening during surgery due to its heavier weight, which consists of a plurality of switching heads.

The present invention provides a universal direction medical positioning structure with a switching head and a fixing rod detachable from each other. The universal direction medical positioning structure can provide interchangeable clamping-based fixing rod and insertion-based fixing rod, which is unattainable by a conventional universal direction medical positioning structure that provides no alternative to the way of coupling the fixing rod to the switching head.

To achieve the above and other functions and effects, the present invention provides a universal direction medical positioning structure, comprising: a positioning unit, comprising: a connecting rod having a first end portion and a second end portion; a support connectable to the first end portion of the connecting rod, comprising at least an arm, and provided with a plurality of positioning elements; and a spherical connecting element coupled to the second end portion of the connecting rod; a switching head provided therein with a hemispherical cavity for receiving the spherical connecting element; a fixing rod having a third end portion and a fourth end portion, the third end portion being connectable to the switching head; and a fixing device coupled to the switching head so as to allow the spherical connecting element to be fixed in position to the hemispherical cavity.

Implementation of the present invention at least involves the following inventive steps:

1. The universal direction medical positioning structure includes the spherical connecting element, the switching head, and the fixing device so as to be lightweight and easy to operate.

2. The universal direction medical positioning structure is lightweight and thereby unlikely to loosen, thereby enhancing positioning preciseness.

3. The switching head and the fixing rod of the universal direction medical positioning structure are detachable from each other such that the universal direction medical positioning structure is advantageously provided with the interchangeable clamping-based fixing rod and insertion-based fixing rod.

The features and advantages of the present invention are described in detail in the preferred embodiments of the present invention so as to enable persons skilled in the art to gain insight into the technical disclosure in the present invention and implement the present invention accordingly and readily understand the objectives and advantages of the present invention by making reference to the disclosure contained in the specification, the claims, and the drawings of the present invention.

### BRIEF DESCRIPTION OF THE SEVERAL VIEWS OF THE DRAWINGS

FIG. 1 is an exploded perspective view of a universal direction medical positioning structure in an embodiment according to the present invention;

FIG. 2 is a schematic view of a fourth end portion of a fixing rod of the universal direction medical positioning structure in another embodiment according to the present invention;

FIG. 3 is an assembled cross-sectional view of the universal direction medical positioning structure in an embodiment according to the present invention;

FIG. 4 is a schematic view of a switching head of the universal direction medical positioning structure in another embodiment according to the present invention;

FIG. 5 is an exploded perspective view of the universal direction medical positioning structure in another embodiment according to the present invention;

FIG. 6 is a perspective view of a second fixing fastener of the universal direction medical positioning structure in an embodiment according to the present invention;

FIG. 7 is a schematic view of a switching head of the universal direction medical positioning structure in another embodiment according to the present invention;

FIG. 8A is an assembled cross-sectional view of a second fixing fastener and a switching head of the universal direction medical positioning structure in an embodiment according to the present invention;

FIG. 8B is an exploded cross-sectional view of a second fixing fastener and a switching head of the universal direction medical positioning structure in an embodiment according to the present invention; and

FIG. 8C is an assembled cross-sectional view of the universal direction medical positioning structure in another embodiment according to the present invention.

### DETAILED DESCRIPTION OF THE INVENTION

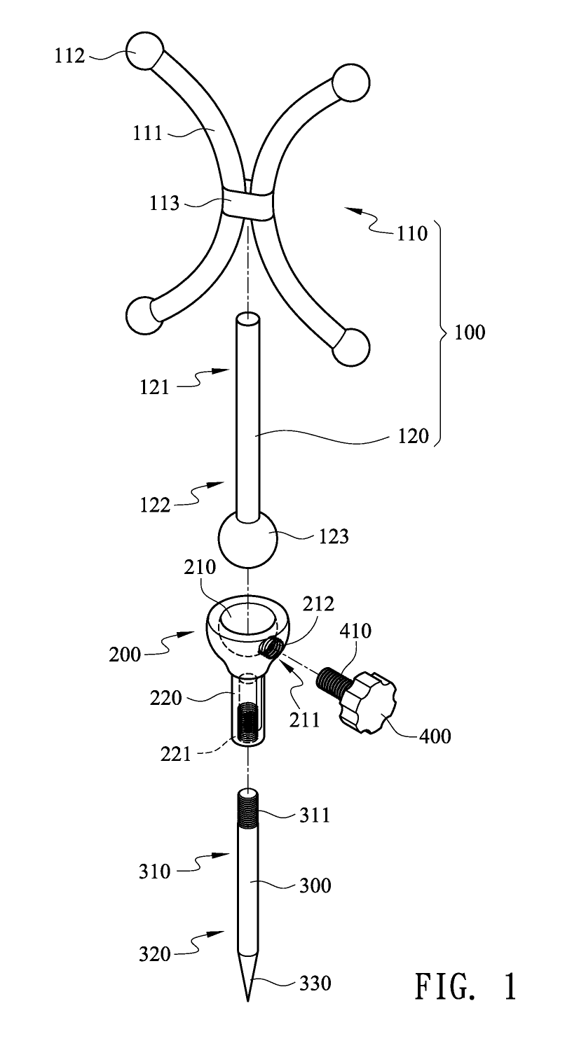

Please refer to FIG. 1 for an exploded perspective view of a universal direction medical positioning structure in an embodiment according to the present invention. As shown in the drawing, the universal direction medical positioning structure comprises a positioning unit 100, a switching head 200, a fixing rod 300, and a fixing device, wherein the fixing device as shown in FIG. 1 is a first fixing fastener 400, and the first fixing fastener 400 is a screw.

Referring to FIG. 1, the positioning unit 100 comprises a support 110, a connecting rod 120, and a spherical connecting element 123. The connecting rod 120 has a first end portion 121 and a second end portion 122. The first end portion 121 of the connecting rod 120 is connectable to the support 110. The second end portion 122 of the connecting rod 120 is coupled to the spherical connecting element 123.

Referring to FIG. 1, the support 110 comprises at least one arm 111, and the arms 111 are connected to a fulcrum 113 in junction. The fulcrum 113 is connectable to the first end portion 121 of the connecting rod 120. The top end of each of the arms 111 is coupled to at least one positioning element 112. The positioning elements 112 and the arms 111 are arranged on a common plane or arranged to form a three-dimensional structure, so as to prevent the positioning elements 112 from being shielded by another object or instrument and enhance positioning precision. The positioning elements 112 must be able to reflect optical signals so as to provide the reflected optical signals to an optical positioning device (not shown) for determining the spatial coordinates of a lesion. Hence, the positioning elements 112 are reflective spheres, infrared reflective bodies, or spheres, each having a reflective marking.

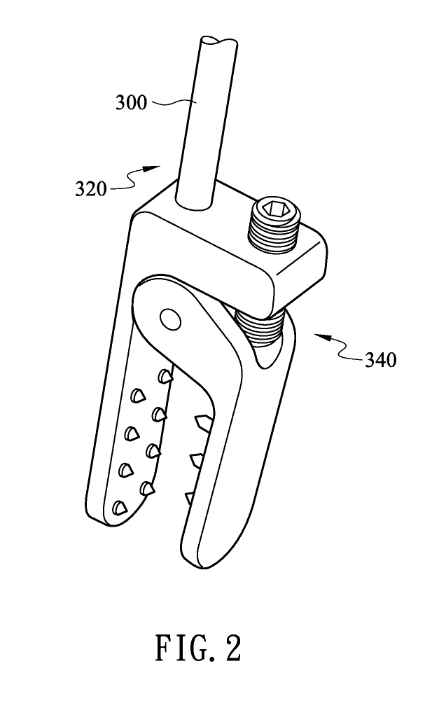

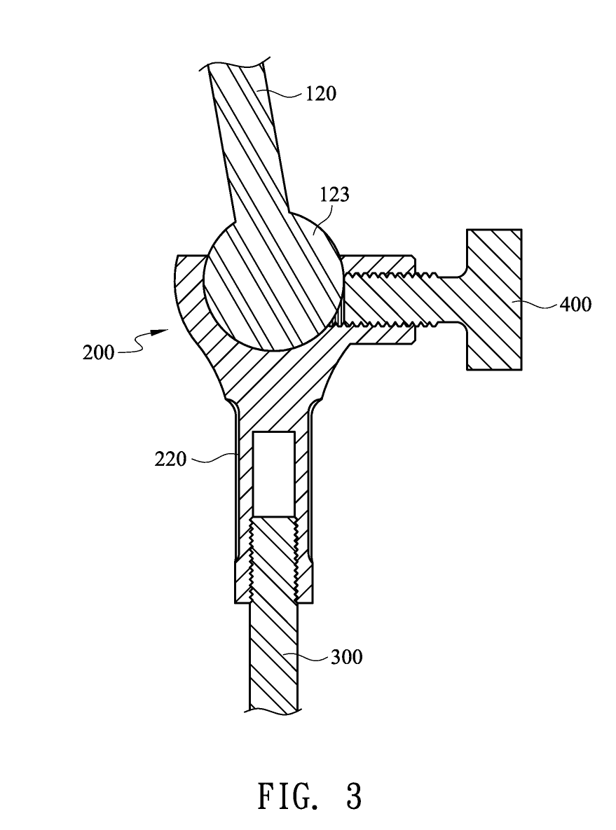

With reference to FIG. 1, the switching head 200 is provided therein with a hemispherical cavity 210 for receiving the spherical connecting element 123 coupled to the second end portion 122 of the connecting rod 120. The fixing rod 300 has a third end portion 310 and a fourth end portion 320. The third end portion 310 is separable from or connectable to the switching head 200 (see FIG. 3). The fourth end portion 320 has a nail-shaped structure 330 replaceable by a clamp-shaped structure 340 (as shown in FIG. 2).

Referring to FIG. 1, the switching head 200 is further provided with a hollow post 220 coupled to the bottom of the switching head 200. A first internal thread 221 is provided on the inner surface of the hollow post 220. A first external thread 311 is provided on the surface of the third end portion 310 of the fixing rod 300. The switching head 200 and the fixing rod 300 are configured to engage with each other, using the first external thread 311 and the first internal thread 221. Also, the switching head 200 is provided with a tapped hole 211 in communication with the hemispherical cavity 210 of the switching head 200. A second internal thread 212 is provided on the inner surface of the tapped hole 211. A second external thread 410 is provided on a portion of the surface of the first fixing fastener 400. The second external thread 410 is configured to fit with the second internal thread 212 of the tapped hole 211 of the switching head 200.

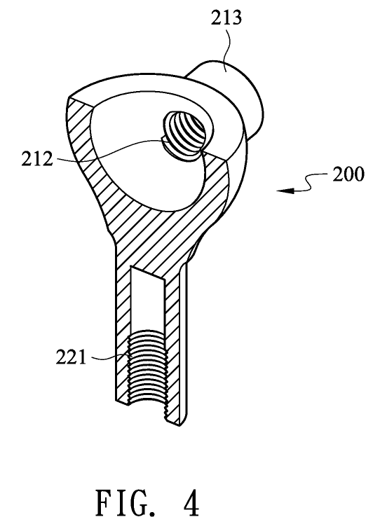

Referring to FIG. 4, in another embodiment, the switching head 200 is further provided with a protruding post 213 protruding therefrom and communicating with the hemispherical cavity 210 of the switching head 200. The second internal thread 212 is provided on the inner surface of the protruding post 213 and is configured to fit with the second external thread 410 of the first fixing fastener 400. Referring to FIG. 3, the first fixing fastener 400 is screwed to the switching head 200, so as for the first fixing fastener 400 to abut against the spherical connecting element 123 coupled to the connecting rod 120, and as a consequence, the spherical connecting element 123 is fixed in position to the hemispherical cavity 210 of the switching head 200 and prevented from rotating.

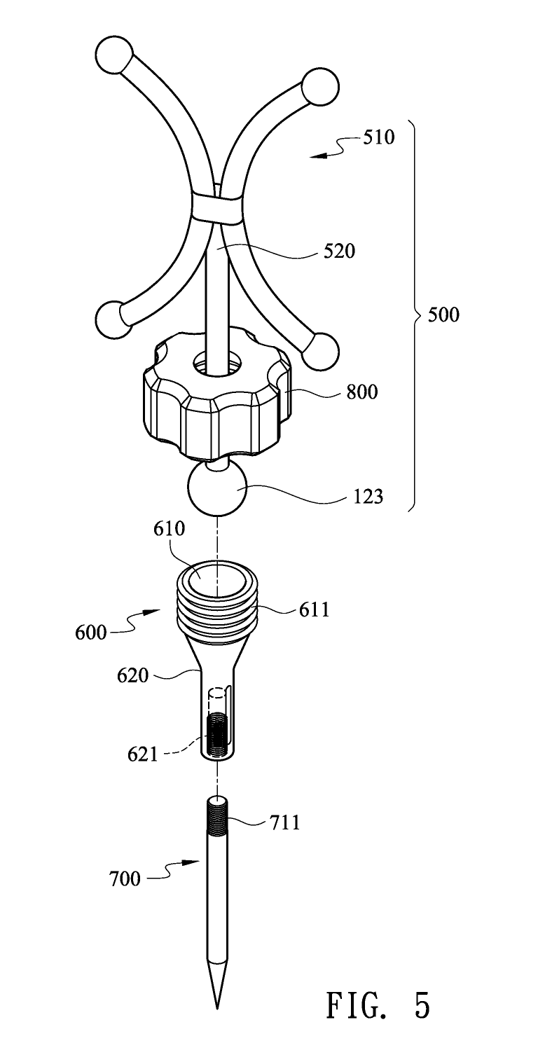

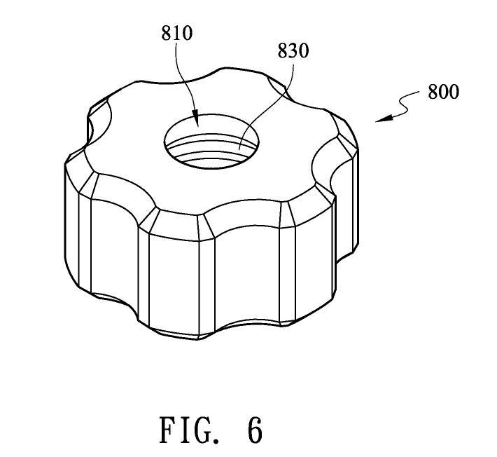

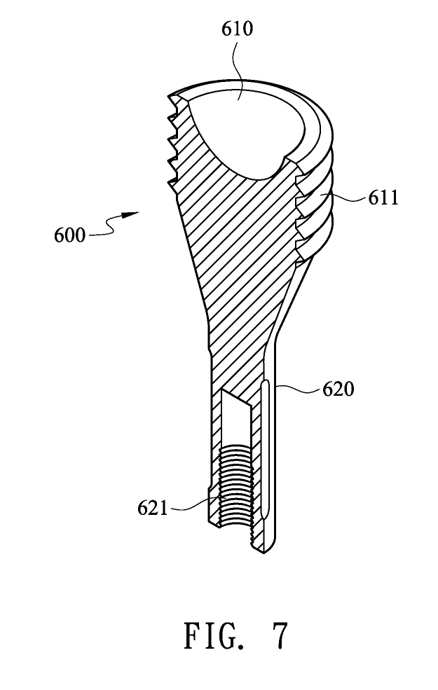

Referring to FIG. 5, in another embodiment, a third external thread 611 is provided on the outer surface of a switching head 600 (see also FIG. 7), and the fixing device is a second fixing fastener 800 penetrated by a connecting rod 520 of a support 510. Referring to FIG. 6, there is shown a perspective view of the second fixing fastener 800 which is a nut. Referring to FIG. 5 and FIG. 6, the second fixing fastener 800 is provided therein with an opening 810, and the inner surface of the opening 810 corresponds in shape to the profile of the spherical connecting element 123 coupled to the connecting rod 520.

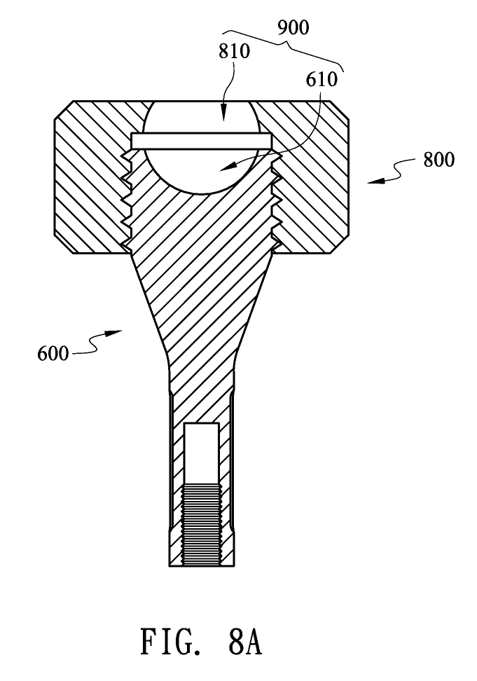

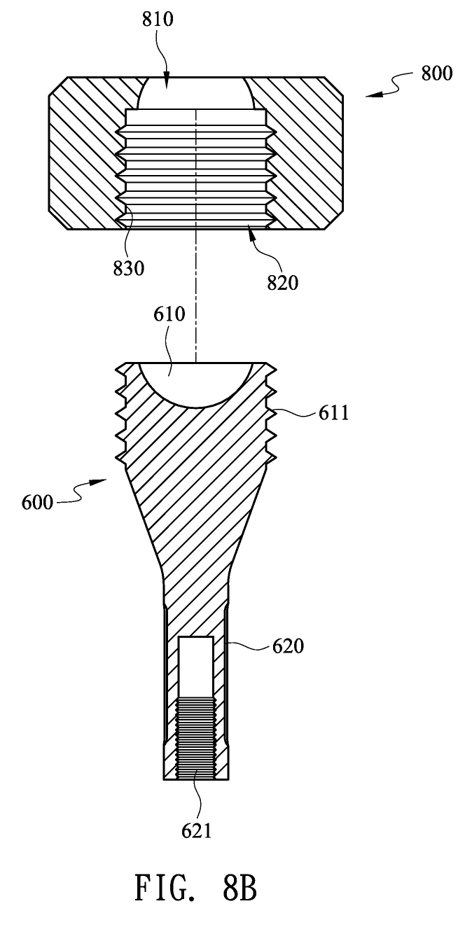

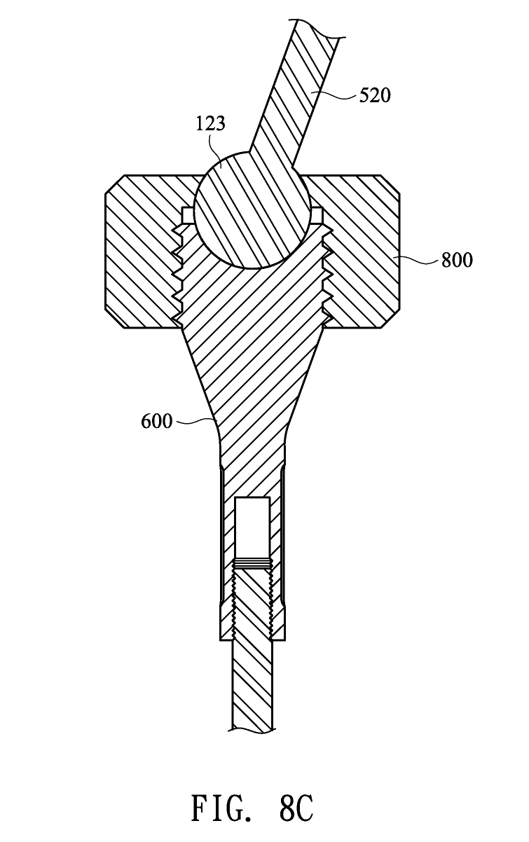

Referring to FIG. 5 and FIG. 8A, upon the coupling of the second fixing fastener 800 and the switching head 600, the opening 810 of the second fixing fastener 800 and a hemispherical cavity 610 of the switching head 600 together form a receiving space 900. Referring to FIG. 8B, the second fixing fastener 800 is further provided therein with a matching hole 820 in communication with the opening 810, and a third internal thread 830 is provided on the inner surface of the matching hole 820. As shown in FIG. 8A through FIG. 8C, the third internal thread 830 on the inner surface of the second fixing fastener 800 can fit with a third external thread 611 on the surface of the switching head 600, thus allowing the second fixing fastener 800 to be engaged with the switching head 600. In consequence, the spherical connecting element 123 of the connecting rod 520 is fixed in position to the receiving space 900.

In conclusion, the orientation of the positioning units 100, 500 can be easily adjusted in the following manner. First, the spherical connecting element 123 inside the hemispherical cavity 610 of the switching heads 200, 600 is rotated freely until the desirable orientation of the positioning units 100, 500 is achieved. Then, the spherical connecting element 123 is fastened to the hemispherical cavity 610 by the fixing devices 400, 800 so as to prevent the positioning units 100, 500 from rotating.

The foregoing embodiments are provided to illustrate and disclose the technical features of the present invention so as to enable persons skilled in the art to understand the disclosure of the present invention and implement the present invention accordingly, and are not intended to be restrictive of the scope of the present invention. Hence, all equivalent modifications and variations made to the foregoing embodiments without departing from the spirit and principles in the disclosure of the present invention should fall within the scope of the invention as set forth in the appended claims.

What is claimed is:

1. A universal direction medical positioning structure comprising:
a positioning unit comprising: a connecting rod having a first end portion and a second end portion; a support connectable to the first end portion of the connecting rod, comprising at least one arm, and provided with a plurality of positioning elements; and a spherical connecting element coupled to the second end portion of the connecting rod;
a switching head provided therein with a hemispherical cavity for receiving the spherical connecting element;
a fixing rod having a third end portion and a fourth end portion, the third end portion being connectable to the switching head; and
a fixing device coupled to the switching head so as to allow the spherical connecting element to be fixed in position to the hemispherical cavity.

2. The universal direction medical positioning structure of claim 1, wherein the support comprises a plurality of arms commonly connected to a fulcrum that is configured to connect to the first end portion of the connecting rod, and a top end of each of the arms is coupled to at least one positioning element.

3. The universal direction medical positioning structure of claim 2, wherein the positioning elements and the arms are arranged on a common plane.

4. The universal direction medical positioning structure of claim 2, wherein the positioning elements and the arms are arranged to form a three-dimensional structure.

5. The universal direction medical positioning structure of claim 1, wherein the positioning element has a reflective marking.

6. The universal direction medical positioning structure of claim 1, wherein the positioning element is a reflective sphere or infrared reflective body.

7. The universal direction medical positioning structure of claim 1, wherein the fourth end portion has a nail-shaped structure or a clamp-shaped structure.

8. The universal direction medical positioning structure of claim 1, wherein the switching head is provided with a hollow post coupled to a bottom of the switching head, and a first internal thread is provided on an inner surface of the hollow post.

9. The universal direction medical positioning structure of claim 8, wherein the third end portion of the fixing rod is provided with a first external thread for fitting with the first internal thread of the hollow post.

10. The universal direction medical positioning structure of claim 1, wherein the switching head is provided with a tapped hole in communication with the hemispherical cavity and the tapped hole is provided with a second internal thread, while the fixing device is a first fixing fastener provided with a second external thread for fitting with the second internal thread of the tapped hole.

11. The universal direction medical positioning structure of claim 10, wherein the first fixing fastener is a screw.

12. The universal direction medical positioning structure of claim 1, wherein the switching head is provided with a protruding post protruding therefrom and communicating with the hemispherical cavity, and a second internal thread is provided on an inner surface of the protruding post, wherein the fixing device is a first fixing fastener, and a second external thread is provided on a portion of a surface of the first fixing fastener for fitting with the second internal thread.

13. The universal direction medical positioning structure of claim 12, wherein the first fixing fastener is a screw.

14. The universal direction medical positioning structure of claim 1, wherein a third external thread is provided on an outer surface of the switching head, wherein the fixing device is a second fixing fastener penetrated by the connecting rod, provided therein with an opening having an inner surface corresponding in shape to a profile of the spherical connecting element and combining with the hemispherical cavity to form a receiving space, and provided therein with a matching hole in communication with the opening, the matching hole having an inner surface provided thereon with a third internal thread for fitting with the third external thread to thereby allow the spherical connecting element to be fixed in position to the receiving space.

15. The universal direction medical positioning structure of claim 14, wherein the second fixing fastener is a nut.
